(19) United States
(12) Patent Application Publication
(10) Pub. No.: US 2023/0417736 A1
(43) Pub. Date: Dec. 28, 2023

(54) MONITORING MEMBRANE PROTEIN TRAFFICKING FOR DRUG DISCOVERY AND DRUG DEVELOPMENT

(71) Applicant: UNIVERSITY OF HOUSTON SYSTEM, Houston, TX (US)

(72) Inventors: Bradley K. McConnell, Houston, TX (US); Arfaxad Reyes Alcaraz, Boston, MA (US)

(73) Assignee: UNIVERSITY OF HOUSTON SYSTEM, Houston, TX (US)

(21) Appl. No.: 18/037,702

(22) PCT Filed: Nov. 1, 2021
(86) PCT No.: PCT/US2021/057525

§ 371 Date: May 18, 2023

Related U.S. Application Data
(60) Provisional application No. 63/115,827, filed on Nov. 19, 2020.

(51) Int. Cl. G01N 33/50; G01N 21/76; G01N 33/58

(52) U.S. Cl. CPC G01N 33/5008; G01N 21/763; G01N 33/581; G01N 2500/10

(57) ABSTRACT

Embodiments of the present disclosure pertain to systems for use in screening at least one binding agent for binding to at least one cell membrane protein. The systems include one or more cells that include the cell membrane protein. The cell membrane protein is genetically engineered to express a first peptide capable of generating a luminescent signal upon interaction with a second peptide. The systems may also include the second peptide. Additional embodiments of the present disclosure pertain to methods of utilizing the systems to screen at least one binding agent for binding to at least one cell membrane protein.

### CROSS-REFERENCE TO RELATED APPLICATIONS

This application claims priority to U.S. Provisional Patent Application No. 63/115,827, filed on Nov. 19, 2020. The entirety of the aforementioned application is incorporated herein by reference.

### BACKGROUND

Current methods of screening potential binding agents for binding to cell membrane receptors have numerous limitations, including a lack of reagent universality, high costs of imaging equipment, and confounding background fluorescence. Numerous embodiments of the present disclosure address the aforementioned limitations.

### SUMMARY

Embodiments of the present disclosure pertain to systems for use in screening at least one binding agent for binding to at least one cell membrane protein. In some embodiments, the systems of the present disclosure include one or more cells that include the cell membrane protein. In some embodiments, the cell membrane protein is genetically engineered to express a first peptide capable of generating a luminescent signal upon interaction with a second peptide. In some embodiments, the first peptide is separated from the cell membrane protein by a flexible amino acid linker, where a majority of the peptide residues include glycine residues, serine residues, or combinations thereof. In some embodiments, the systems of the present disclosure also include the second peptide. In some embodiments, the first peptide includes Small fragment of Nano Luciferase (SmBiT) and the second peptide includes Large fragment of Nano Luciferase (LgBit).

Additional embodiments of the present disclosure pertain to methods of utilizing the systems of the present disclosure to screen at least one binding agent for binding to at least one cell membrane protein. In some embodiments, the methods of the present disclosure include: (a) associating the binding agent with a cell that includes the cell membrane protein, where the cell membrane protein is genetically engineered to express a first peptide capable of generating a luminescent signal upon interaction with a second peptide; (b) detecting a presence or an absence of an internalization of the cell membrane protein by the presence or absence of the luminescent signal, respectively; and (c) correlating the presence or absence of the internalization to the binding or lack of binding of the binding agent to the cell membrane protein, respectively.

### DESCRIPTION OF THE DRAWINGS

FIG. 1A depicts a system for screening a binding agent for binding to a cell membrane protein.

FIG. 1B illustrates a method of screening a binding agent for binding to a cell membrane protein.

FIGS. 2A-F illustrate G protein-coupled receptor (GPCR) internalization via early endosomes. FIG. 2A provides a schematic of the overall concept of a structural complementation assay to monitor internalization in real-time living cells. FIG. 2B is a schematic of a structural complementation system based on Nano Luciferase showing the LgBiT (Large fragment of Nano Luciferase) and SmBiT (Small fragment of Nano Luciferase). FIG. 2C shows different internalization rates across different GPCRs. Galanin receptors were stimulated using 10 μM Galanin (GAL₁ and GAL₂ receptors) whereas, in the case of chemokine receptor 4 (CCR4), 100 ng/ml of SDF-1α was used. FIG. 2D shows dose-dependent internalization of human β2AR in early endosomes in cardiomyocytes using different concentrations of epinephrine. FIG. 2E shows validation of the GPCR internalization assay using 10 μM of endocytosis inhibitors, cmpd101, PiTStop, and Dynasore. FIG. 2F shows dose-response curve stimulation for internalization of β2AR. The results are expressed as mean±s.e.m. of three experiments performed in triplicate; each triplicate was averaged before calculating the s.e.m. The corresponding Z′ factor was 0.63.

FIGS. 3A-B show monitoring in real-time the internalization and recycling of a prototypical GPCR. Shown in FIGS. 3A-B are recycling of β2AR in HEK293 cells using 10 μM epinephrine; Number “1” indicates the receptor being removed from the plasma membrane, Number “2” corresponds to the receptor being localized in early endosomes, and Number “3” indicates the washout of the ligand and return of the receptor to the cell surface. The arrows indicate the time at which the cells were treated with the corresponding ligand. The results are expressed as mean±s.e.m. of three experiments performed in triplicate; each triplicate was averaged before calculating the s.e.m.

FIGS. 4A-F show schematics of the Proximity Ligation Assay (PLA). Two primary antibodies recognize specific proteins in the cell. FIG. 4A shows β2AR-SmBiT and Early Endosome Antigen 1 (EEA1). FIG. 4D shows LgBiT-FYVE and Early Endosome Antigen 1. In both cases, secondary antibodies coupled with oligonucleotides (PLA probes) bind to the primary antibodies. When the PLA probes are in close proximity, connector oligos join the PLA probes and become ligated. The resulting closed, circular DNA template becomes amplified by DNA polymerase. Then, complementary detection oligos coupled to fluorochromes hybridize to repeating sequences in the amplicons. PLA signals are detected by fluorescent microscopy as discrete spots and provide the intracellular localization of the protein or protein interaction. Confocal images were obtained from the PLA of the early endosomes (FIGS. 4B, 4C, 4E, and 4F). FIG. 4B shows the PLA assay using antibodies targeting β2AR and EEA1 in the absence of ligand (vehicle control). FIG. 4C shows the PLA assay using the same antibodies as in FIG. 4C but where the sample was incubated for 5 minutes in the presence of 10 μM epinephrine. FIG. 4E shows the PLA assay using antibodies targeting the LgBiT-FYVE and EEA1 in the absence of ligand (vehicle control). FIG. 4F shows the PLA assay using the same antibodies as in FIG. 4E but where the sample was incubated for 5 minutes in the presence of 10 μM epinephrine. High magnification (scale bar=10 μm) or FIG. 4C and FIG. 4F are shown in the inserts to the far right, respectively.

FIG. 5 illustrates visualization of receptor localization in HEK293 cells using a bioluminescence LV200 Olympus microscope. HEK293 cells were transfected with β2AR-SmBiT and LgBiT-FYVE. The luminescence images were acquired after the addition of the luciferase substrate, furimazine, and 10 μM epinephrine (final concentration) by capturing total luminescence for 2 minutes at each of the indicated times. Scale bar represents 20 μm.

FIGS. 6A-6B provides illustrations of non-GPCR HER 2 receptors. FIG. 6A shows a schematic representation for monitoring internalization of the HER2 receptor. FIG. 6B shows HER2 receptor time course internalization of cells expressing the receptor treated with 10 μM (final concentration) human epidermal growth factor (hEGF). The arrow indicates the time when the cells were treated with the virus. The results are expressed as mean±s.e.m. of three experiments performed in triplicate; each triplicate was averaged before calculating the s.e.m. The corresponding Z′ factor was 0.70.

FIGS. 7A-C illustrate membrane protein internalization by binding with two monoclonal antibodies. FIG. 7A provides a schematic of a membrane protein being internalized by the binding with an antibody via early endosomes. FIGS. 7B-1 and 7B-2 show the membrane protein (FAM19A5 Isoform II) being internalized when cells expressing FAM19A5 Isoform II were treated with 10 nM and 100 nM of two antibodies (these antibodies are currently under development). 1 μM IgG antibody was used as a control. The top panel shows antibody A and the bottom panel shows antibody B. FIG. 7C shows dose-response curves for both antibodies. The corresponding EC50 value for antibody A was 7.08±0.2 nM (Z′ factor of 0.92) and for antibody B (Z′ factor of 0.943) was 10.54±0.5 nM. The arrow indicates the time when the cells were treated with the virus. The results are expressed as mean±s.e.m. of three experiments performed in triplicate; each triplicate was averaged before calculating the s.e.m.

FIGS. 8A-8B show monitoring SARS-CoV2 infection in real-time in living HEK293 cells. FIG. 8A shows a schematic representation of how the Lentivirus expressing the SARS-CoV2 Spike protein is internalized by binding with Angiotensin-Converting Enzyme 2 (ACE2) via early endosomes. FIG. 8B shows real-time monitoring of viral entry into HEK293 cells expressing human ACE2 when treated with the Lentivirus. The arrow indicates the time when the cells were treated with the virus. The results are expressed as mean±s.e.m. of three experiments performed in triplicate; each triplicate was averaged before calculating the s.e.m. The corresponding Z′ score was 0.50.

FIG. 9 shows amino acid sequences of the constructs used to monitor receptor internalization and SARS-CoV2 infection.

FIG. 10 shows amino acid sequences of the constructs used to monitor antibody mediated internalization of FAM19A5 isoform II.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

The advent of high-throughput screening (HTS) has enabled successful unbiased drug-discovery and fostered the development of novel therapies. Arguably, the most fruitful targets in HTS platforms have been membrane proteins that comprise 22% of the proteins encoded by the genome and are targeted by 60% of the approved drugs available today. Almost half of these drugs are directed at the rhodopsin-like class A G protein-coupled receptor (GPCR) superfamily. Many of these receptors have underlying roles in a myriad of diseases, including cancer, heart disease, diabetes, metabolic diseases, pulmonary diseases, renal diseases, hepatic disease, drug addiction, alcoholism, chronic pain, autoimmune diseases, mood disorders, and mental illness. Therefore, membrane proteins represent a goldmine of targets that must be screened to fully exploit their rich therapeutic potential.

HTS platforms for plasma membrane receptors have had success due to reliable cell-based systems for monitoring the diversity of downstream messenger pathways, such as cAMP signaling, calcium mobilization, and Rho GTPase activation. However, in most cases, these assays are highly idiosyncratic and consequently require the development of specialized protocols. HTS becomes particularly challenging for those receptors that are of great therapeutic interest but have non-canonical signaling or remain uncharacterized. Thus, plasma membrane trafficking is the single universal feature of membrane receptor protein regulation.

The reasons why HTS trafficking screens are not more often utilized include a lack of reagent universality, high costs of imaging equipment, and confounding background fluorescence that, in many instances, requires sophisticated deconvolution algorithms to identify subpopulations of membrane proteins. Accordingly, a need exists for more effective methods of screening potential binding agents to membrane receptors. Numerous embodiments of the present disclosure address the aforementioned need.

In some embodiments, the present disclosure pertains to systems for use in screening at least one binding agent for binding to at least one cell membrane protein. In some embodiments, the systems of the present disclosure include one or more cells that include at least one cell membrane protein. In some embodiments, the systems of the present disclosure also include a second peptide.

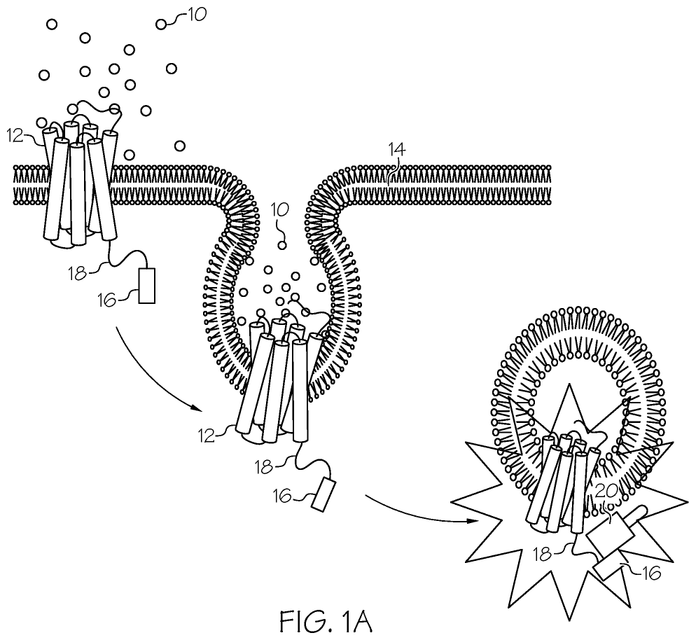

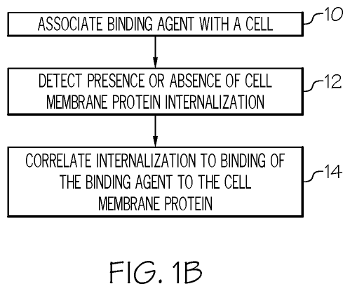

In some embodiments illustrated in FIG. 1A, the systems of the present disclosure include one or more cells that include at least one cell membrane protein 12. In this embodiment, cell membrane protein 12 is embedded in cellular membrane 14 and genetically engineered to express a first peptide 16, which is separated from the cell membrane protein by a flexible amino acid linker 18. In some embodiments, the systems of the present disclosure also include candidate binding agents 10 and second peptides 20.

In operation, candidate binding agents 10 are associated with cell membrane protein 12. Thereafter, internalization occurs if binding agent 10 binds to cell membrane protein 12. Such internalization is detectable by the presence of a luminescent signal, which occurs when first peptide 16 interacts with second peptide 20.

In additional embodiments, the present disclosure pertains to methods of screening at least one binding agent for binding to at least one cell membrane protein. In some embodiments, the methods of the present disclosure utilize the systems of the present disclosure. In some embodiments illustrated in FIG. 1B, the methods of the present disclosure include: associating a binding agent with a cell that includes at least one cell membrane protein that is genetically engineered to express a first peptide capable of generating a luminescent signal upon interaction with a second peptide (step 10); detecting a presence or absence of an internalization of the cell membrane protein by detecting the presence or absence of the luminescent signal, respectively (step 12); and correlating the presence or absence of the internalization to the binding or lack of binding of the binding agent to the cell membrane protein, respectively (step 14).

The methods and systems of the present disclosure can have numerous embodiments. For instance, the methods and systems of the present disclosure can be utilized in various manners to screen numerous binding agents for binding to numerous cell membrane proteins of numerous cells.

### Binding Agents

The methods and systems of the present disclosure may be utilized to screen numerous binding agents for binding to cell membrane proteins. For instance, in some embodiments, the binding agents include, without limitation, small molecules, macromolecules, peptides, proteins (including large proteins), antibodies, aptamers, drugs, drug candidates, odors, pheromones, hormones, neurotransmitters, catecholamines, growth factors, fatty acids, proteases, antivirals, and combinations thereof.

In some embodiments, the binding agent is a drug or a drug candidate for a disease. As such, in some embodiments, the methods and systems of the present disclosure may be utilized to screen or evaluate drugs or drug candidates for the treatment or prevention of various diseases.

In some embodiments, the disease includes, without limitation, cancer, diabetes, metabolic diseases, pulmonary diseases, renal diseases, hepatic disease, drug addiction, alcoholism, chronic pain, autoimmune diseases, mood disorders, heart disease, mental illness, microbial infections, HIV, eye diseases, or combinations thereof. In some embodiments, the disease includes microbial infections, such as viral infections.

In some embodiments, the disease includes a microbial infection caused by a coronavirus. In some embodiments, the coronavirus includes, without limitation, severe acute respiratory syndrome coronavirus (SARS-CoV), severe acute respiratory syndrome-related coronavirus (SARSr-CoV), human coronavirus 229E (HCoV-229E), human coronavirus NL63 (HCoV-NL63), human coronavirus 0C43 (HCoV-0C43), human coronavirus HKU1 (HCoV-HKU1), Middle East respiratory syndrome-related coronavirus (MERS-CoV), severe acute respiratory syndrome-related coronavirus 2 (SARS-CoV2), variants of SARS-CoV2, or combinations thereof. In some embodiments, the disease includes COVID-19.

In some embodiments, the disease includes a cancer. In some embodiments, the cancer includes, without limitation, tracheal cancer, lung cancer, bronchial cancer, epithelial cancer, blood cancer, breast cancer, melanoma, ovarian cancer, leukemia, lymphomas, prostate cancer, bladder cancer, colon cancer, gliomas, sarcomas, glioblastoma, or combinations thereof. In some embodiments, the cancer includes lung cancer.

### Cell Membrane Proteins

The methods and systems of the present disclosure may be utilized to screen binding agents for binding to numerous types of cell membrane proteins. For instance, in some embodiments, the cell membrane protein includes, without limitation, cell membrane receptors, G-protein coupled receptors (GPCRs), plasma membrane proteins, human beta 2 adrenergic receptors, viral receptors such as Angiotensin Converting Enzyme 2, HER 2 receptors, or combinations thereof. In some embodiments, the cell membrane protein is an endogenously expressed cell membrane protein.

### First and Second Peptides

In some embodiments, the cell membrane proteins of the present disclosure are genetically engineered to express a first peptide. In some embodiments, the first peptide is capable of generating a luminescent signal upon interaction with a second peptide. In some embodiments, the second peptide is not embedded with the cell membrane. In some embodiments, the luminescent signal is the only readout to detect the presence or absence of an internalization of the cell membrane.

The methods of the present disclosure may utilize various first and second peptides. For instance, in some embodiments, the first peptide includes Small fragment of Nano Luciferase (SmBiT). In some embodiments, SmBiT includes a sequence of VTGYRLFEEIL (SEQ ID NO:1). In some embodiments, SmBIT includes a sequence that shares at least 65% sequence identity to SEQ ID NO:1. In some embodiments, SmBIT includes a sequence that shares at least 70% sequence identity to SEQ ID NO:1. In some embodiments, SmBIT includes a sequence that shares at least 75% sequence identity to SEQ ID NO: 1 . In some embodiments, SmBIT includes a sequence that shares at least 80% sequence identity to SEQ ID NO: 1. In some embodiments, SmBIT includes a sequence that shares at least 85% sequence identity to SEQ ID NO:1. In some embodiments, SmBIT includes a sequence that shares at least 90% sequence identity to SEQ ID NO: 1. In some embodiments, SmBIT includes a sequence that shares at least 95% sequence identity to SEQ ID NO:1. In some embodiments, SmBIT includes a sequence that shares at least 99% sequence identity to SEQ ID NO:1.

In some embodiments, SmBIT includes a peptide no longer than 11 amino acids. In some embodiments, SmBIT includes a peptide longer than 11 amino acids. In some embodiments, SmBIT includes a peptide no greater than 1.4 KDa.

In some embodiments, the second peptide includes Large fragment of Nano Luciferase (LgB it). In some embodiments, LgBiT includes a sequence of VFTLEDFVGDWEQTAAYNLDQVLEQGGVSSLLQNLAVSVTPIQRIVRSGENALKIDIHVI IPYEGLSADQMAQIEEVFKVVYPVDDHHFKVILPYGTLVIDGVTPNMLNYFGRPYEGIA VFDGKKITVTGTLWNGNKIIDERLITPDGSMLFRVTINS (SEQ ID NO:2). In some embodiments, LgBiT includes a sequence that shares at least 65% sequence identity to SEQ ID NO:2. In some embodiments, LgBiT includes a sequence that shares at least 70% sequence identity to SEQ ID NO:2. In some embodiments, LgBiT includes a sequence that shares at least 75% sequence identity to SEQ ID NO:2. In some embodiments, LgBiT includes a sequence that shares at least 80% sequence identity to SEQ ID NO:2. In some embodiments, LgBiT includes a sequence that shares at least 85% sequence identity to SEQ ID NO:2. In some embodiments, LgBiT includes a sequence that shares at least 90% sequence identity to SEQ ID NO:2. In some embodiments, LgBiT includes a sequence that shares at least 95% sequence identity to SEQ ID NO:2. In some embodiments, LgBiT includes a sequence that shares at least 99% sequence identity to SEQ ID NO:2.

In some embodiments, LgBiT includes a protein no greater than 18 KDa. In some embodiments, LgBiT includes a protein with no lower affinity towards the SmBiT (SEQ ID NO:1) than 150 μM.

In some embodiments, the first peptide is separated from the cell membrane protein by a flexible amino acid linker. In some embodiments, the second peptide includes a flexible amino acid linker. In some embodiments, a majority of the flexible amino acid linker residues include glycine residues, serine residues, or combinations thereof. In some embodiments, at least 90% of the flexible amino acid linker residues include glycine residues, serine residues, or combinations thereof. In some embodiments, at least 85% of the flexible amino acid linker residues include glycine residues, serine residues, or combinations thereof. In some embodiments, at least 80% of the flexible amino acid linker residues include glycine residues, serine residues, or combinations thereof. In some embodiments, at least 75% of the flexible amino acid linker residues include glycine residues, serine residues, or combinations thereof. In some embodiments, at least 70% of the flexible amino acid linker residues include glycine residues, serine residues, or combinations thereof. In some embodiments, at least 65% of the flexible amino acid linker residues include glycine residues, serine residues, or combinations thereof.

In some embodiments, the flexible amino acid linker includes at least 18 residues. In some embodiments, the flexible amino acid linker includes at least 15 residues. In some embodiments, the flexible amino acid linker includes at least 10 residues. In some embodiments, the flexible amino acid linker includes at least 5 residues.

In some embodiments, the flexible amino acid linker includes, without limitation, GSSGGGGSGGGGSSGGAQGNS (SEQ ID NO:3), GNSGSSGGGGSGGGGSSG (SEQ ID NO:4), GSSGGGGSGGGGSSG (SEQ ID NO:5), GSSGGGGSGGGGSSGGAQGNS (SEQ ID NO:6), a sequence that shares at least 65% sequence identity to any one of SEQ ID NOS: 3-6, or combinations thereof.

In some embodiments, the flexible amino acid linker shares at least 70% sequence identity to any one of SEQ ID NOS: 3-6. In some embodiments, the flexible amino acid linker shares at least 75% sequence identity to any one of SEQ ID NOS: 3-6. In some embodiments, the flexible amino acid linker shares at least 80% sequence identity to any one of SEQ ID NOS: 3-6. In some embodiments, the flexible amino acid linker shares at least 85% sequence identity to any one of SEQ ID NOS: 3-6. In some embodiments, the flexible amino acid linker shares at least 90% sequence identity to any one of SEQ ID NOS: 3-6. In some embodiments, the flexible amino acid linker shares at least 95% sequence identity to any one of SEQ ID NOS: 3-6. In some embodiments, the flexible amino acid linker shares at least 99% sequence identity to any one of SEQ ID NOS: 3-6.

In some embodiments, the second peptide also includes a cell membrane insertion domain. In some embodiments, the cell membrane insertion domain inserts onto an internalized cell membrane in a pH dependent manner. In some embodiments, the cell membrane insertion domain is separated from the second peptide by a flexible amino acid linker.

In some embodiments, the cell membrane insertion domain includes a sequence of MQKQPTWVPDSEAPNCMNCQVKFTFTKRRHHCRACGKVFCGVCCNRKCKLQYLEKE ARVCVVCYETISK (SEQ ID NO: 7). In some embodiments, the cell membrane insertion domain shares at least 65% sequence identity to SEQ ID NO: 7. In some embodiments, the cell membrane insertion domain shares at least 70% sequence identity to SEQ ID NO: 7. In some embodiments, the cell membrane insertion domain shares at least 75% sequence identity to SEQ ID NO: 7. In some embodiments, the cell membrane insertion domain shares at least 80% sequence identity to SEQ ID NO: 7. In some embodiments, the cell membrane insertion domain shares at least 85% sequence identity to SEQ ID NO: 7. In some embodiments, the cell membrane insertion domain shares at least 90% sequence identity to SEQ ID NO: 7. In some embodiments, the cell membrane insertion domain shares at least 95% sequence identity to SEQ ID NO: 7. In some embodiments, the cell membrane insertion domain shares at least 99% sequence identity to SEQ ID NO: 7.

In some embodiments, the cell membrane proteins and second peptides of the present disclosure are expressed in cells by plasmids. In some embodiments, the plasmids utilize low expression promoters in order to prevent non-specific interactions. In some embodiments, the low expression promoters include Herpes simplex virus (HSV) thymidine kinase promoters.

### Cells

The systems and methods of the present disclosure may utilize various types of cells. Suitable cells generally include cells that express the cell membrane proteins of the present disclosure.

In some embodiments, the cells include live cells. In some embodiments, the cells are cultivated in vitro, such as in a petri dish. In some embodiments, the cells are genetically engineered to express cell membrane proteins with a first peptide (e.g., SmBiT) that is capable of generating a luminescent signal upon interaction with a second peptide (e.g., a protein fragment, such as LgBit).

In some embodiments, the cells express the second peptide (e.g., a protein fragment, such as LgBit). In some embodiments, the cells express the second peptide such that the second peptide is covalently linked to an early endosome marker.

The methods and systems of the present disclosure may utilize various types of cells. For instance, in some embodiments, the cells include, without limitation, Human Embryonic Kidney Cells, Chinese Ovary Hamster Cells, Mouse embryonic fibroblasts, Human Epithelial Carcinoma, Human T-Cell Leukemia, Human breast cancer cell lines, human colorectal adenocarcinoma cell lines, Human neuroblastoma cell lines, Human Hepatoma cell lines, Human promyelocytic leukemia cell lines, Glioma cell lines, Pancreas cancer cell lines, PC-3 prostate cell lines, Human Stem Cells, COS-1, COS-7, AC16 Cardiomyocytes, HeLa cells, BEAS-2B epithelial cells, Spodoptera Frugiperda insect cells, SK-UT-1 Uterus cell lines, and combinations thereof.

Association of Binding Agents with Cells

Various methods may be utilized to associate binding agents with cells in order to screen the binding of the binding agents to cell membrane proteins of cells. For instance, in some embodiments, the association occurs through the addition of binding agents to the cells. In some embodiments, the association occurs by mixing the binding agents with the cells. In some embodiments, the association lacks any washing steps. In some embodiments, the association occurs without the use of automated equipment. In some embodiments, the association occurs without the need for reagent washes or automated equipment.

### Detecting Internalization of Cell Membrane Proteins

Internalization of cell membrane proteins is generally characterized by the presence of a luminescent signal. Similarly, the absence of internalization of cell membrane proteins is generally characterized by the absence of luminescent signals.

Various methods may also be utilized to detect the internalization of cell membrane proteins after association with binding agents. For instance, in some embodiments, the internalization is detected by detecting an endocytosis of a cell membrane protein. In some embodiments, the internalization is detected by detecting a receptor-mediated endocytosis of a cell membrane protein.

In some embodiments, the internalization of a cell membrane protein is detected by monitoring the internalization of the cell membrane protein through an increase in luminescence emitted by the cell membrane protein after internalization. In some embodiments, the luminescence includes bioluminescence.

In some embodiments, the increase in luminescence occurs when a first peptide fused to a cell membrane protein interacts with a second peptide (e.g., protein fragment) associated with an early endosome marker or an internalized cell membrane. In some embodiments, the first peptide is SmBiT, and the second peptide (e.g., a protein fragment) is LgBit.

In some embodiments, the second peptide (e.g., protein fragment) is covalently linked to a membrane protein. In some embodiments, the first peptide is covalently attached to an early endosome marker or an internalized cell membrane.

In some embodiments, the internalization is detected in real-time. In some embodiments, the absence of internalization is correlated to the lack of binding of a binding agent to a cell membrane protein. In some embodiments, the presence of internalization is correlated to a binding of the binding agent to a cell membrane protein.

### Screening

The methods and systems of the present disclosure may be utilized to screen binding agents against cell membrane proteins in various manners. For instance, in some embodiments, a single binding agent is screened against a single type of cell membrane protein associated with one or more cells. In some embodiments, a single binding agent is screened against a plurality of different types of cell membrane proteins associated with one or more cells. In some embodiments, a plurality of different binding agents is screened against a single type of cell membrane protein associated with one or more cells. In some embodiments, a plurality of different binding agents is screened against a plurality of different types of cell membrane proteins associated with one or more cells.

The methods and systems of the present disclosure may be utilized to screen binding agents against cell membrane proteins in various environments. For instance, in some embodiments, the screening occurs in vitro. In some embodiments, the screening occurs in real-time. In some embodiments, the screening occurs in an array. In some embodiments, the screening occurs through high throughput screening, preclinical screening, automated and fast screening, or combinations thereof.

### Applications and Advantages

The methods and systems of the present disclosure can have numerous applications and advantages. For instance, in some embodiments, the methods and systems of the present disclosure can be utilized to characterize the pharmacological properties of one or more binding agents. In some embodiments, the methods and systems of the present disclosure can be utilized to screen different types of binding agents (e.g., hormones, drug candidates, and antibodies) for binding to a cell membrane on a same platform. Moreover, in some embodiments, the methods and systems of the present disclosure can be utilized to perform real-time screening of binding agents in live cells. In some embodiments, the methods and systems of the present disclosure mimic physiological conditions of cell membrane proteins by evaluating the binding of endogenously expressed cell membrane proteins to binding agents.

In some embodiments, the methods and systems of the present disclosure provide a powerful, unrestricted, and universal technology of drug discovery and drug development that is based on trafficking properties of plasma membrane proteins. On the other hand, current HTS technologies are restricted and not universal for the following reasons: lack of reagent universality, high costs of imaging equipment, and confounding high background fluorescence that complicates data interpretation.

In some embodiments, the methods and systems of the present disclosure can be utilized for plasma membrane protein drug discovery. In most cases, current plasma membrane protein drug discovery assays are highly idiosyncratic and consequently require specialized protocol development. HTS becomes especially challenging for those receptors that are therapeutically relevant but have non-canonical signaling or remain uncharacterized. Thus, the methods and systems of the present disclosure facilitate drug discovery through detecting plasma membrane protein trafficking, which can be the single universal feature of membrane receptor protein regulation.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. An Assay to Monitor Membrane Proteins Trafficking for Drug Discovery and Drug Development

Internalization of membrane proteins plays a key role in many physiological functions. However, highly sensitive and versatile technologies are lacking to study such processes in real-time living systems. Here, Applicant describes an assay based on bioluminescence able to quantify membrane receptor trafficking for a wide variety of internalization mechanisms such as GPCR internalization and recycling, antibody-mediated internalization, and SARS-CoV2 viral infections. This study represents an alternative drug discovery tool to accelerate the drug development for a wide range of physiological processes, such as cancer, neurological, cardiopulmonary, metabolic, and infectious diseases including COVID-19.

In this Example, Applicant hypothesized that, due to the high sensitivity of bioluminescence, Applicant could devise an assay using blue light emission to monitor a wide variety of internalization processes by targeting the early endosomes and using NanoLuc Binary Technology (NanoBiT). Bioluminescence resonance energy transfer (BRET) between a Renilla luciferase-inserted GPCR and a GFP10-fused FYVE domain was previously developed to measure GPCR internalization. Therefore, Applicant replaced the BRET pair with a split luciferase (NanoBiT) and added flexible amino acid linkers in between.

Here, Applicant reports a methodology based on bioluminescence, produced by the fragment complementation of Nano Luciferase (NLuc). This assay allows for quantifying real-time membrane protein internalization and recycling in living systems by using bioluminescence. Moreover, this method can universally be applied to monitor the internalization of a wide range of membrane proteins and be used in the elucidation of novel molecular mechanisms as well as the development of therapeutic agents for cancer, cardiopulmonary and infectious diseases including COVID-19.

### Example 1.1. GPCR Trafficking

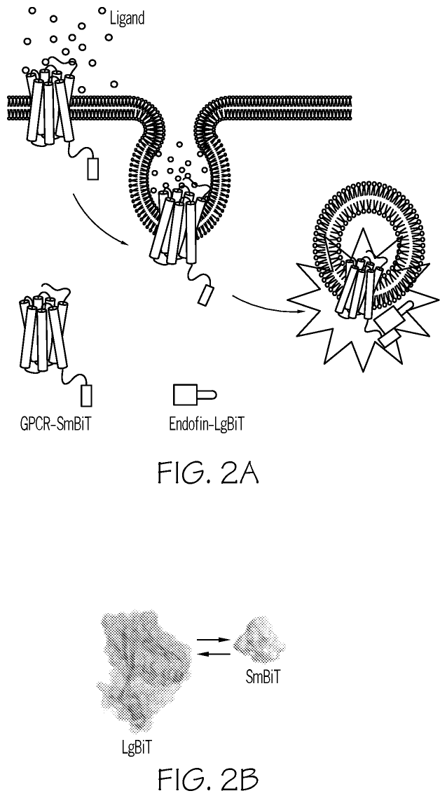

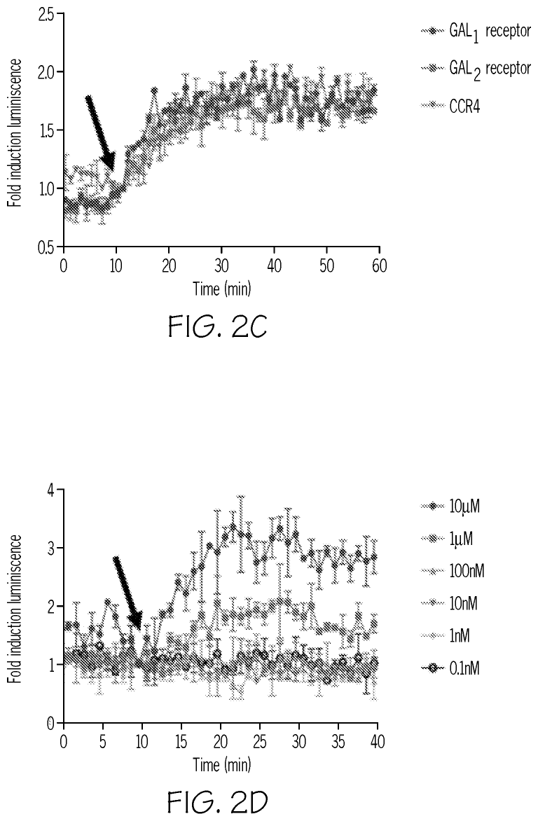

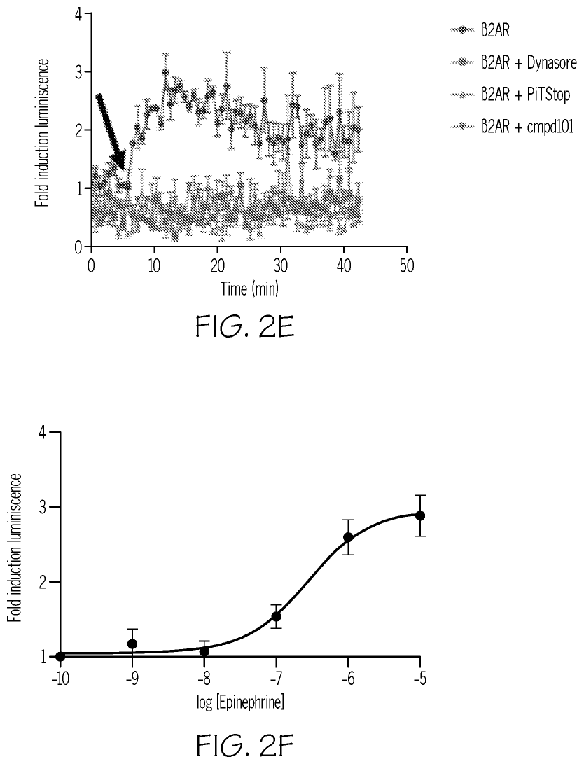

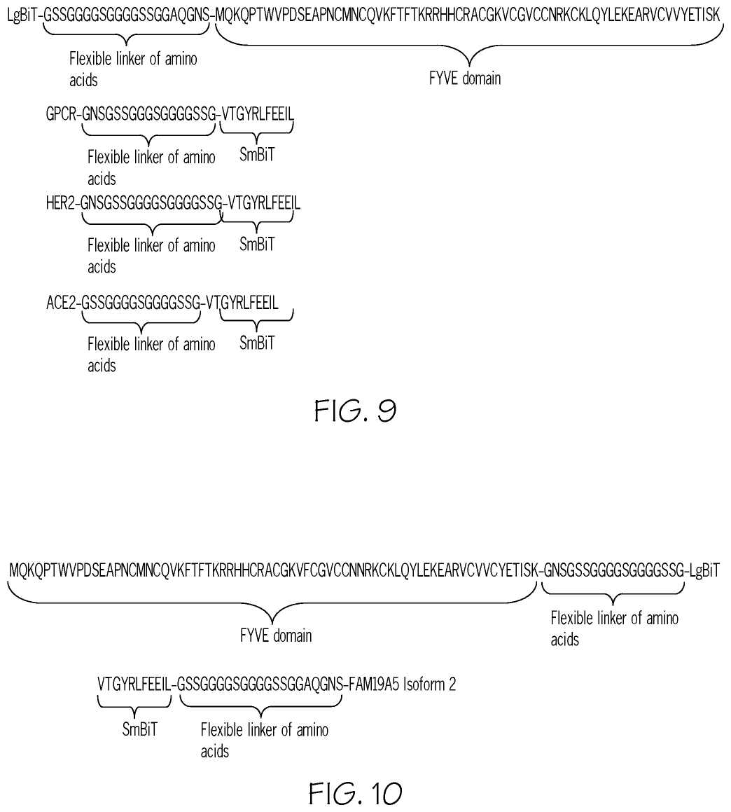

Applicant initially focused on studying the different internalization rates of class A G-protein coupled receptors (GPCRs). For this, Applicant first established a set of GPCRs involved in a wide variety of physiological functions and diseases such as Galanin (GAL receptors), Chemokine receptors (CCR), and β-adrenergic receptors ((βAR). Applicant devised a strategy to monitor how the GPCR bound to its corresponding ligand is removed from the cell surface, subsequently localized in early endosomes, and then finally recycled into the cell membrane. To accomplish this, Applicant covalently linked a small fragment of NLuc (SmBiT) to the C-terminal of the receptor, where a flexible linker is located between the two proteins (FIGS. 9-10). This linker was designed to be composed mainly of glycine and alanine amino acids to provide flexibility and not to alter the pharmacological properties of the native receptor (FIGS. 2A-2B).

To detect light emission when the receptor is being localized at the early endosomes, Applicant focused on the interaction between the early endosome and the FYVE zinc finger domain, where the FYVE zinc finger domain is named after the four cysteine-rich proteins: Fab 1 (yeast orthologue of PlKfyve), YOTB, Vac 1 (vesicle transport protein), and EEA1(Early Endosome Antigen 1).

FYVE domains bind phosphatidylinositol 3-phosphate from early endosomes, in a manner that is dependent on its metal ion coordination and basic amino acids. This FYVE domain inserts into cell membranes in a pH-dependent manner, where it is composed of two small beta hairpins (or zinc knuckles) and followed by an alpha helix. Additionally, the FYVE finger binds two zinc ions where this FYVE finger has eight potential zinc coordinating cysteine positions and is characterized by having basic amino acids around the cysteines. To achieve this, the gene of the FYVE domain of the human Endofin (residues from Q739 to K806) was synthesized and covalently attached by molecular cloning into a large fragment of NLuc (LgBiT) at the N-termini (FIGS. 2A-2B).

Applicant observed an increase in luminescence in the β-adrenergic receptor 2 (β2AR) and two additional GPCRs upon agonist-mediated stimulation (FIG. 2C), reaching a maximum in luminescence intensity at 30 minutes after agonist treatment. In the case of β2AR, Applicant used epinephrine to cause epinephrine-mediated sequestration of receptors from the plasma membrane and translocate them into early endosomes. This increased the blue luminescent signal in a concentration-dependent manner in response to the agonist (FIG. 2D).

Regarding the quantitative pharmacological analysis of GPCRs, HEK293 cells were treated with increasing concentrations of agonists. The time course graph displayed an increase in normalized luminescence over time with increasing agonist concentrations. The curve analysis of this response demonstrates a clear concentration-dependent response of human β2AR internalization (FIGS. 2D-F).

To show the specificity of the internalization processes, Applicant then validated the approach by using inhibitors targeting the vital elements involved in receptor endocytosis (FIG. 2E). Internalization of GPCRs was inhibited by using the GPCR kinase 2/3 inhibitor (Cmpd101), a selective inhibitor of clathrin-mediated endocytosis (PiTStop) and dynamin inhibitor (Dynasore), consistent with the role that β-arrestins have in agonist-dependent and mediated internalization of GPCRs. Finally, a dose-response stimulation curve was obtained (FIG. 2D) to quantify the internalization potency for the epinephrine at the β2AR (FIG. 2F).

### Example 1.2. Assessing Recycling and Forward Trafficking of a Prototypical GPCR

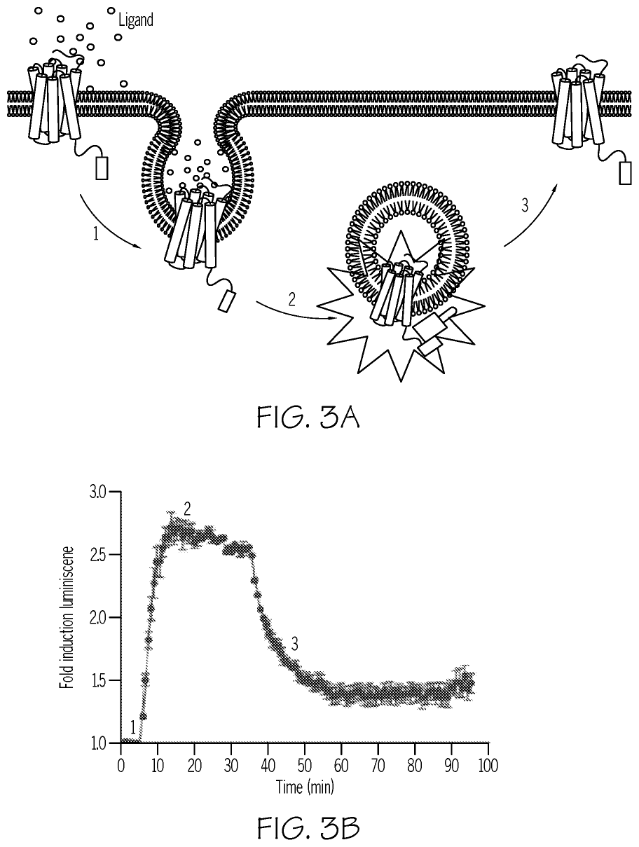

By using the same approach, Applicant was able to study not only GPCR internalization but also receptor recycling (FIG. 3A). After treating the cells expressing β2AR-SmBiT with the final concentration of 10 μM epinephrine, the internalization process occurred. This internalization process was observed by a rapid increase in luminescence, indicating the receptor was being removed from the cell surface (FIG. 3A, number-1). This was then followed by a maximum in the luminescent signal being reached, suggesting the receptor was localized at the endosome (FIG. 3A, number-2).

After a few minutes of signal stability, Applicant took the plate out of the luminometer, removed the medium containing the ligand and replaced it with fresh medium in the absence of epinephrine, and continued measuring the luminescence signal. Applicant observed a gradual decay of signal in the absence of ligand, indicating the ligand-receptor complex was being dissociated and the receptor and localized back to the plasma membrane (FIG. 3A, number-3). It is interesting to note that the β2AR recycling back to the cell surface was slightly slower as compared to its internalization kinetics.

### Example 1.3. Luminescent Signal is Originated from Early Endosomes

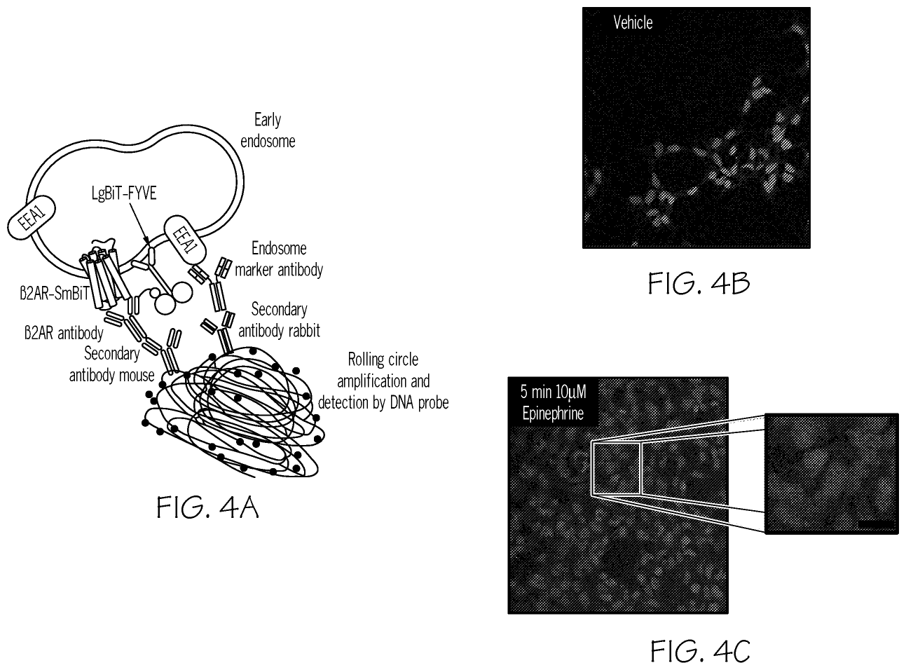

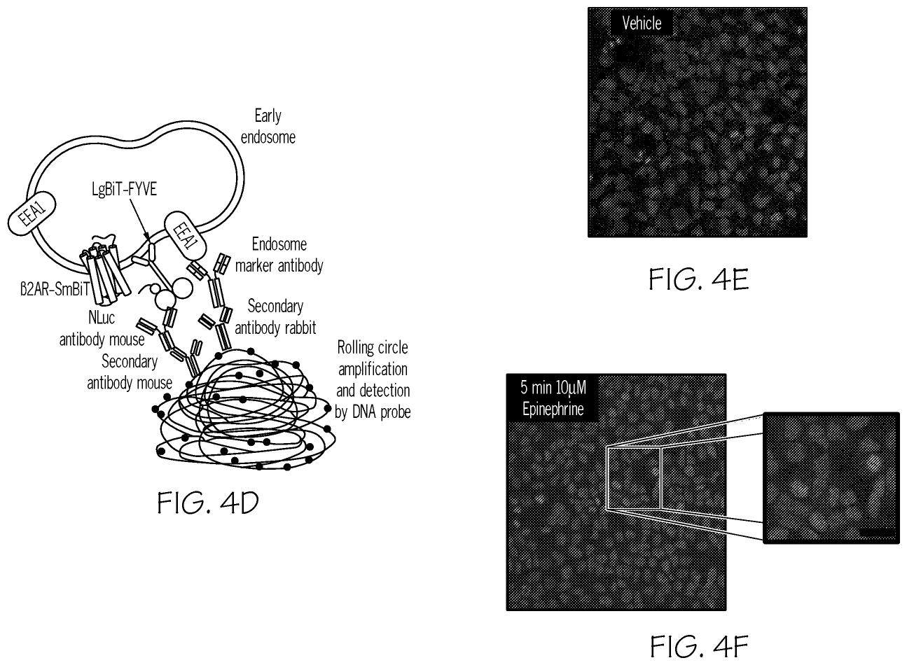

To demonstrate that the luminescent signal is generated from early endosomes, Applicant performed a proximity ligation assay (PLA). The PLA assay is a powerful tool to detect close proximity (about 30 nm) between two entities with high specificity and sensitivity. In this case, the protein targets Applicant used were (1) NLuc, attached to the FYVE domain, (2) EEA1, at the early endosome, and (3) the β2AR (FIG. 4). Applicant then used two primary antibodies, raised in different species (rabbit and mouse), to detect two unique protein targets (NLuc and EEA1 or β2AR and EEA1). A pair of oligonucleotide-labeled secondary antibodies (PLA probes) were bound to the primary antibodies (FIGS. 4A-D).

Next, hybridizing connector oligos joined the PLA probes. If the PLA probes were in close proximity to each other and, the ligation process formed a closed circle, serving as the DNA template required for the rolling-circle amplification (RCA). This allowed up to a 1000-fold amplified signal that was still tethered to the PLA probe, allowing localization of the signal. Lastly, labeled oligos hybridized to the complementary sequences within the amplicon which were then visualized and quantified as discrete red spots (PLA signals) by microscopy image analysis (FIGS. 3C and 3F).

### Example 1.4. Live Cell Imaging: GPCR Internalization

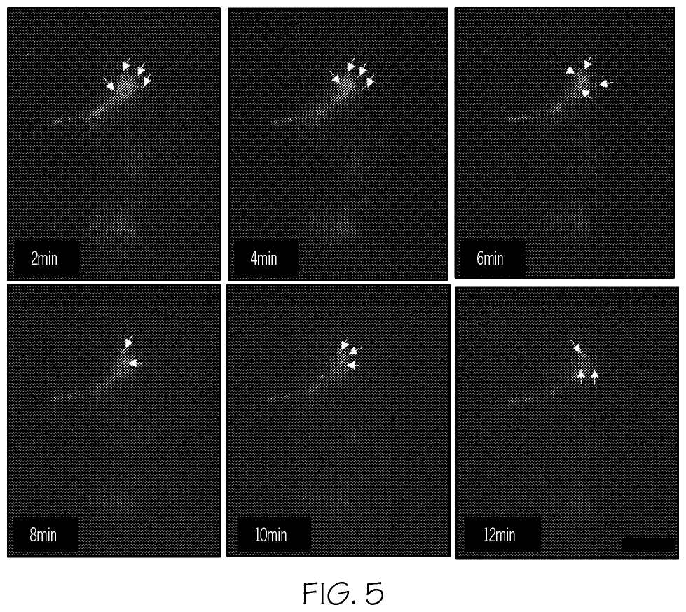

To visualize the GPCR internalization in live cells, Applicant used a bioluminescent microscope to characterize the receptor localization within the cells. This trafficking visualization is unique in that being able to measure receptor internalization in living cells and in real-time by using luminescence. Most other technologies that quantify receptor internalization do not offer information on spatiotemporal live-cell imaging since they are not performed in real-time and living systems. Thus, Applicant was able to visualize the trafficking of a prototypical GPCR (β2AR) in real-time and in living cells (FIG. 5). This trafficking was observed as small luminescent spots moving through the cytosol (highlighted in arrows).

After ligand addition, Applicant recorded the β2AR internalization by capturing total luminescence every 2 minutes. Applicant observed a decrease in signal intensity along with the experiment as a consequence of furimazine (NLuc substrate) depletion

### Example 1.5. Non-GPCR Membrane Receptor Internalization

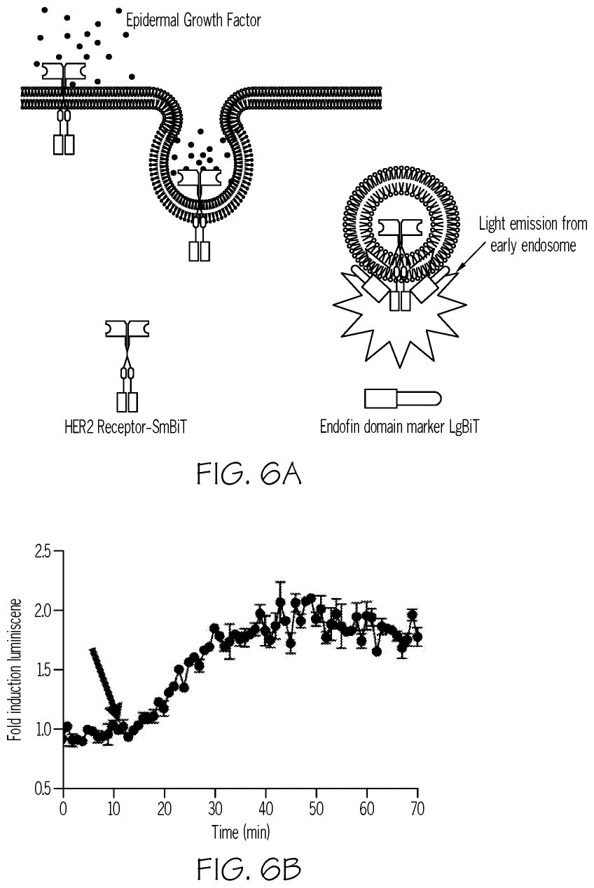

To continue exploring potential applications of Applicant's technology, Applicant next used a prototypical non-GPCR receptor, the human epidermal growth factor receptor 2 (EGFR2), also referred to as the HER2 receptor (FIG. 6A). In this study, Applicant was able to observe that the internalization rate was slightly slower compared to some GPCRs, suggesting that agonists that activate membrane receptors follow similar internalization kinetics (FIG. 6B).

### Example 1.6. Antibody-Mediated Internalization

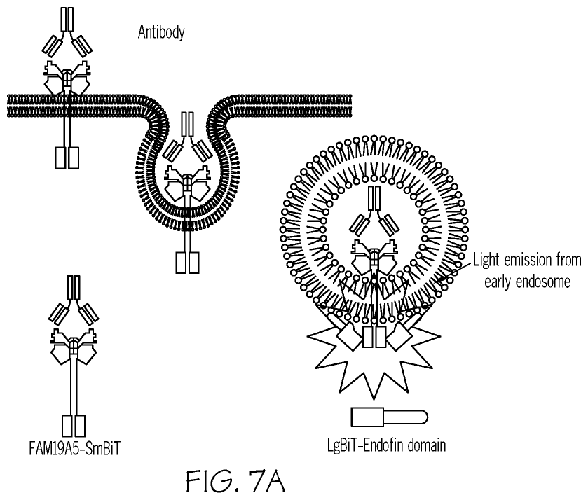

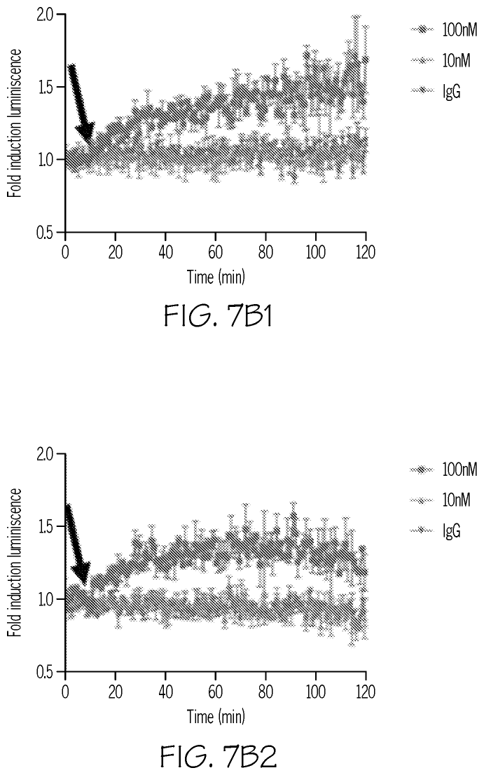

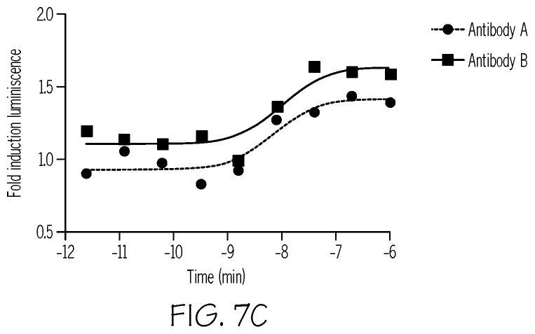

Being able to measure antibody-mediated internalization is currently one of the most exciting receptor trafficking mechanisms to study in medicine. To test the versatility of Applicant's methodology, Applicant set up a strategy to monitor antibody-mediated internalization. Applicant used a membrane protein recently discovered termed FAM19A5 Isoform II (also called TAFAS). It has been described that FAM19A5 plays a key role in neurological disorders. Applicant decided to use FAM19A5 Isoform II as a prototypical system to study antibody-mediated internalization using two antibodies currently under development. Applicant tagged FAM19A5 Isoform II with SmBiT at the N-termini. SmBiT-FAM19A5 isoform II was co-expressed with the FYVE domain tagged with the LgBiT at the N-termini (FIG. 7A). In this experiment, Applicant observed slower internalization kinetics than observed for the GPCRs (FIG. 2). This slower internalization kinetics suggested a slow conformational change in the receptor induced by the binding with the antibody (FIGS. 7B-1 and 7B-2). Applicant achieved to quantify and compare the internalization potencies for the two antibodies, highlighting that antibody A was slightly more potent than antibody B (FIG. 7C).

### Example 1.7. SARS-CoV2 Viral Entry

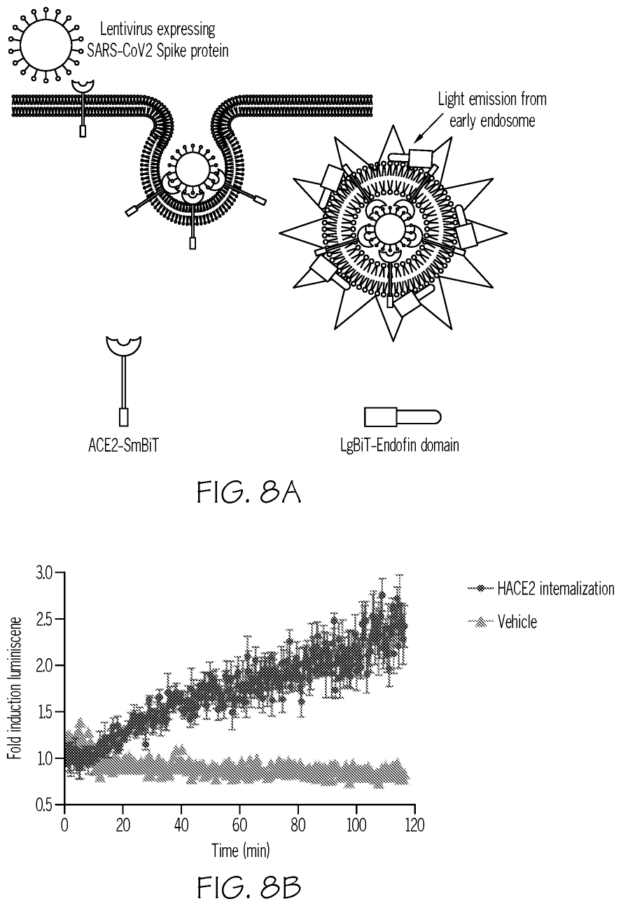

Finally, since internalization also encompasses viral infection, Applicant devised a system to monitor, in real-time, viral entry into the cell by using the SARS-CoV2 Spike protein as a prototypical system of viral infection (FIG. 8A). In this experimental approach, Applicant observed viral entry into the cells is mediated by early endosomes and that its kinetics is also much slower as compared to ligand-activated receptors (FIG. 2).

The viral entry trafficking, using the SARS-CoV2 Spike protein (FIG. 8B), was similar to the antibody-mediated receptor trafficking (FIGS. 7B-1, 7B-2 and 7C). In ongoing experiments, Applicant is exploring whether new variants (delta and mu) of SARS-CoV2, increase the internalization rate and kinetics of the Angiotensin Converting Enzyme 2 (ACE2) internalization.

### Example 1.8. Discussion

In this study, Applicant described several strategies to accurately quantify membrane receptor internalization across different systems by setting up a structural complementation assay based on NLuc. Other studies have been reported in the literature that also used the structural complementation of NLuc but adapted to different physiological contexts such as GPCR dimerization and oligomerization. The approach of structural complementation has great potential in drug discovery and structural biology. It has been successfully applied for several applications in biological research.

At the beginning of this study, Applicant aimed to develop a universal drug discovery tool that can be applied to nearly any membrane receptor. After studying different physiological processes where membrane receptors are involved, Applicant conclude that almost any membrane protein expressed at the cell surface undergoes internalization, and therefore, Applicant hypothesize that it would be possible to monitor the activity of a particular receptor by observing, in real-time, its trafficking in living systems.

For this purpose, Applicant used the FYVE domain of endofin since this domain binds to early endosomes. Applicant covalently linked the FYVE domain to the large fragment of NLuc (FIG. 9).

As the first application of Applicant's methodology, Applicant decided to explore how internalization occurs across class A GPCRs, the largest class of GPCRs, accounting for nearly 85% of the GPCR genes that encode for rhodopsin-like receptors (i.e., β2AR), olfactory and orphan receptors. Applicant were able to observe that the internalization kinetics reached a maximum within about 10 minutes after ligand stimulation and the fold induction of bioluminescence was between 2 and 3, depending on the GPCR (FIG. 2C).

To validate Applicant's methodology, Applicant pretreated the cells expressing the system with internalization inhibitors (FIG. 2E). Applicant was able to verify that the luminescent signal was abolished in the presence of those inhibitors. Such observation suggests that the increase in the luminescent signal after ligand stimulation is originated from the GPCR internalization.

One of the advantages of a real-time assay is that Applicant can study different conditions in the same well of the assay plate. To illustrate this, Applicant monitored the GPCR internalization and recycling in the same sample (FIGS. 3A-B).

Moreover, this technique is useful in the de-orphanization of GPCRs, especially in cases where the receptor signaling is unknown, as well as in the development and characterization of agonists and antagonists for GPCRs.

The potential application of this internalization assay also can be applied to other classes of membrane receptors beyond the study and characterization of the GPCRs—where GPCRs account for approximately 35% of the Federal Drug Administration (FDA) approved drugs. In addition to GPCRs, Applicant set out to apply the assay to receptor tyrosine kinases (RTKs), another class of membrane receptors. Specifically, Applicant applied the methodology to the human epidermal growth factor receptor 2 (HER2), a member of the receptor tyrosine-protein kinase ErbB family of receptors that promote cell proliferation and opposes apoptosis.

The application of this technology to the HER2 receptor is highly significant since HER2 is known to have therapeutic importance in breast and ovarian cancers. Thus, Applicant studied HER2 receptor internalization, recycling, or degradation in late endosomes where this membrane receptor trafficking pattern represents pivotal internalization steps towards the development of treatments of HER2 positive cancer patients. Applicant's results demonstrate that Applicant can monitor membrane protein trafficking of RTKs, specifically HER2, using an approach similar to the methodology described for GPCRs.

As further validation regarding the universality of Applicant's membrane protein internalization assay, Applicant applied this technology to monitor antibody-mediated internalization. As such, Applicant studied the Family with sequence similarity 19 (chemokine (C-C motif)-like) member A5 (FAM19A5) receptor protein, a member of the TAFA family a chemokine-like protein that regulates cell proliferation and migration.

Specifically, FAM19A5 is a novel gene with multiple physiological functions (i.e., neurokine, adipokine) recently being discovered. For this membrane protein, Applicant was able to study antibody-mediated internalization of FAM19A5 by two newly develop monoclonal antibodies (FIGS. 7A-B).

In contrast to GPCRs or RTK receptors, antibody-mediated internalization of FAM19A5 displayed much slower kinetics, reaching a maximum of internalization at two hours after antibody treatment. The fold induction was slightly lower than previously seen in GPCRs or RTK receptors, presumably because the binding between the antibody and FAM19A5 induced minor conformational changes on the receptor as compared to the ligand-receptor interactions observed for β2AR and HER2.

Finally, Applicant sought to extend the membrane protein internalization assay to monitor viral infections. Specifically, Applicant set out to monitor SARS-CoV2, the seventh coronavirus known to infect humans and able to cause severe respiratory disease, can cause multiorgan infection and cell tropism in the human body. Moreover, the SARS-CoV2-mediated receptor trafficking can be studied and characterized for drug discovery. Thus, when SARS-CoV2 binds into the cell, the virus/plasma membrane receptor can be monitored as an internalization process mediated by virus particles (FIG. 8A). For this purpose, Applicant produced lentivirus expressing the SARS-CoV2 spike protein.

Applicant then added the viral suspension containing the SARS-CoV2 spike protein to cells expressing the ACE2 receptor and then covalently linked to the small domain of NLuc. This design strategy enabled the simulation of the SARS-CoV2 infection in real-time and in living cells. Applicant's results demonstrate that Applicant can monitor membrane protein trafficking of SARS-CoV2 spike protein with a good signal-noise ratio and where the entire infection process takes approximately three hours and this virus-receptor complex continues to be internalized for some time thereafter. As such, Applicant envisions that this assay will enable the studying of neutralizing antibodies or antivirals. Furthermore, this technology can also be extended to other infection systems using other viruses like those mediated by a GPCR, as in the case of the C-C chemokine receptor type 5 (CCR5) and C-X-C chemokine receptor type 4 (CXCR4), members of the chemokine receptor family, during an HIV-1 infection.

In general, the main advantage of Applicant's method is that the internalization of the receptor can be monitored in real-time and the versatility to study a wide range of internalization mechanisms, ranging from GPCRs, RTKs, antibody to viral entry into the cell. Applicant's assay shows a dynamic range between 1.5 to 3, which was similar to other approaches using the structural complementation of NLuc and slightly lower as compared to other approaches measuring receptor-arrestin interactions in real-time.

Some aspects remain to be studied in Applicant's methodology, such as to evaluate whether if tagging the receptors with the SmBiT alters its native pharmacology and distribution at the plasma membrane as well as expressing the tagged receptors at endogenous levels.

In conclusion, Applicant both theoretically and experimentally illustrated the universality of Applicant's structural complementation approach to study a wide range of membrane protein internalization mechanisms. Furthermore, Applicant defined a comprehensive set of cell-based assays and demonstrated their ability of monitoring membrane protein trafficking. These assays have direct application to drug discovery and drug development. The internalization rates vary dramatically ranging from a few minutes (in the case of GPCRs) to a few hours (in the case of viral entry into the cells). Altogether, a universal methodology such as that illustrated in this work will accelerate drug discovery and drug development for numerous types of diseases that are based on membrane protein trafficking.

### Example 1.9. Methods

The ability to monitor internalization in response to ligand stimulation was assessed in HEK293 cells expressing the corresponding human receptor. In the case of GPCRs, the assay was performed using the corresponding endogenous ligands (i.e., epinephrine).

Cell culture medium and cell culture additives were from Life Technologies. All chemicals were obtained from Sigma-Aldrich (St. Louis, MO, USA) unless otherwise stated. The restriction enzymes were obtained from New England Bio Labs (Ipswich, MA, USA). All ligand peptides were synthesized by AnyGen (Gwangju, Korea). The synthesized peptide purity was greater than 98% as determined by high-performance liquid chromatography analysis. All peptides were dissolved in dimethyl sulfoxide and then diluted in media to the desired working concentrations.

### Example 1.10. NanoBit Technology

The NanoBit starter kit containing the plasmids and the necessary reagents for the development of the structural complementation assays used in this study were obtained from Promega Company (Madison, Wisconsin, USA).

Applicant designed primers to introduce genes of interest into pBiT1.1-C [TK/LgBiT], pBiT2.1-C [TK/SmBiT], pBiT1.1-N [TK/LgBiT] and pBiT2.1-N [TK/SmBiT] vectors. Applicant selected at least one of these three sites as one of the two unique restriction enzymes needed for directional cloning due to the presence of an in-frame stop codon that divides the multi-cloning site. Applicant incorporated nucleotide sequences into the primers to encode the linker residues shown in FIGS. 9-10. For pBiT1.1-C [TK/LgBiT] and pBiT2.1-C [TK/SmBiT] vectors, Applicant made sure that the 5′ primer contained an ATG codon and a potent Kozak consensus sequence (GCCGCCACC). For pBiT1.1-N [TK/LgBiT] and pBiT2.1-N [TK/SmBiT] vectors, Applicant ensured that the 3′ primer contained a stop codon.

Applicant prepared a 1% agarose gel to run the digested DNA plasmid and insert and proceed to cut the corresponding bands. Once the corresponding vector and insert bands were purified, Applicant determined the DNA concentration using a spectrophotometer. Applicant performed DNA ligation to fuse the insert to the recipient plasmid. Applicant prepared ligation reactions of around 100 ng of total DNA including 50 ng of plasmid vector. Applicant set up a recipient plasmid-insert ratio of approximately 1:3. Applicant also set up negative controls in parallel. For instance, ligation of the recipient plasmid DNA without any insert provided information about how much background of undigested or self-ligating recipient plasmid was present.

Applicant picked 3-10 individual bacterial colonies and transferred them into 1 mL of LB medium containing ampicillin (100m/mL) and incubated them for 6 hours. Then, Applicant used 200 μL of bacterial suspension and transferred it to 5 mL of LB medium containing the same concentration of ampicillin and incubated overnight at 37.5° C. with shaking at 200 rpm. Applicant performed miniprep DNA purifications using 5 mL of LB grown overnight following the manufacturer's instructions (Life Technologies). To identify successful ligations, Applicant set up PCR reactions using the DNA obtained from mini-preps as a template with the same primers as during the first PCR used for cloning. Positive clones produced the PCR products with the corresponding insert size. Applicant verified the construct sequence by sequencing using primers.

### Example 1.11. Primary Ligation Assays

For primary ligation assays, HEK293 cells were seeded in an 8 well Lab-Tek II Chamber Slide (Life Technologies) with a density of 2×10 5 cells per well. The next morning, cells were transfected with 200 ng of β2AR-SmBiT and 200 ng of LgBiT-FYVE constructs using Viafect (Promega Corporation). The next day, samples were treated with 10 μM epinephrine final concentration for 5 minutes, and immediately after, cells were incubated with 4% paraformaldehyde for 15 minutes at room temperature. Thereafter, the cells were rinsed with PBS and permeabilized with PBS containing 0.1% Tween 20 (PBST). Then, cells were incubated with blocking buffer (Duolink blocking buffer for PLA) at 37.5° C. for 1 hour and followed by incubation with the primary antibodies, anti-β2AR (Abcam catalog number), anti-NLuc (Abcam catalog number), or anti-Early Endosome Antigen 1 (Abcam catalog number) at 1:1000 by diluting in the Duolink antibody dilution buffer at 4° C. overnight. After three washes (5 minutes each) with PBST, the cells were incubated with PLA probes (PLUS and MINUS PLA probes) in a pre-heated humidity chamber for 1 hour at 37.5 ° C. Three washes (5 minutes each) were then performed. Ligation of the two PLA probes was performed by incubation of the slides in a pre-heated humidity chamber for 30 minutes at 37° C. in the presence of ligase and 1× ligation buffer.

### Example 1.12. Internalization Assay using NanoBit Technology

HEK293 cells were maintained in Dulbecco's modified Eagle's medium (DMEM) supplemented with 10% fetal bovine serum (FBS), 100 U/ml penicillin G, and 100 m/ml streptomycin (Invitrogen; Carlsbad, CA, USA). At 1 day before transfection, the cells were seeded in 96-well plates at a density of 2.5×10⁴ cells per well. A mixture containing 100 ng receptor construct containing the LgBit or SmBit and 50 ng of the Endofin domain containing one of the two domains of Nano luciferase and 0.3 μl Viafect (Promega) was prepared and added to each well. Applicant tested four Endofin-receptor spatial orientations. The one with the highest signal was chosen for further experiments to obtain maximum sensitivity. At 24 hours post-transfection, the medium was aspirated and replaced with 100 μl OPTIMEM (Life Technologies, Grand Island, NY, USA). After a 10 minute incubation, 25 μl substrate (furimazine) was added, and once every minute subsequent luminescence measurements were taken for 5-10 minutes for signal stabilization. A total of 10 μl of ligand, antibody, or viral suspension was then added to each well and luminescence measurements were recorded immediately and once every minute for 1-3 hours (Synergy 2 Multi-Mode Microplate Reader BioTek, Winooski, VT, USA).

### Example 1.13. Lentivirus-Mediated Expression of the Spike Protein of SARS-CoV2

All manipulations were taking place in a biosafety cabinet at all times. HEK293 cells were transfected with the plasmids containing SARS-CoV-2, Wuhan-Hu-1 (GenBank: NC 045512), spike-pseudotyped lentiviral kit (NR-52948, from Bei Resources) designed to generate pseudotyped lentiviral particles expressing the spike (S) glycoprotein gene, as well as luciferase (Luc2) and green fluorescent protein (GFP). Seventy-two hours after transfection, the medium was collected in a 50 ml tube and store at −80 ° C. for further applications. This protocol only requires Biosafety Level 1 (BSL1) conditions and the viruses used in this protocol were replication-defective.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above but is only limited by the claims, including all equivalents of the subject matter of the claims. The disclosures of all patents, patent applications, and publications cited herein are hereby incorporated herein by reference, to the extent that they provide procedural or other details consistent with and supplementary to those set forth herein.

1. A method of screening at least one binding agent for binding to at least one cell membrane protein, said method comprising:
(a) associating the binding agent with a cell comprising the cell membrane protein,
wherein the cell membrane protein is genetically engineered to express a first peptide,
wherein the first peptide is separated from the cell membrane protein by a flexible amino acid linker,
wherein a majority of the flexible amino acid linker residues comprise glycine residues, serine residues, or combinations thereof, and
wherein the first peptide is capable of generating a luminescent signal upon interaction with a second peptide;
(b) detecting a presence or an absence of an internalization of the cell membrane protein after the associating step, wherein the internalization is characterized by the presence of the luminescent signal, and wherein the absence of the internalization is characterized by the absence of the luminescent signal; and
(c) correlating the presence of the internalization to the binding of the binding agent to the cell membrane protein, or correlating the absence of the internalization to the lack of binding of the binding agent to the cell membrane protein.

2. The method of claim 1, wherein the binding agent is selected from the group consisting of small molecules, macromolecules, peptides, proteins, antibodies, aptamers, drugs, drug candidates, odors, pheromones, hormones, neurotransmitters, catecholamines, growth factors, fatty acids, proteases, antivirals, and combinations thereof.

3. The method of claim 1, wherein the binding agent is a drug or a drug candidate for a disease, and wherein the method is utilized to screen or evaluate the drug or the drug candidate for the treatment or prevention of the disease.

4. (canceled)

5. The method of claim 3, wherein the disease is selected from the group consisting of cancer, diabetes, metabolic diseases, pulmonary diseases, renal diseases, hepatic disease, drug addiction, alcoholism, chronic pain, autoimmune diseases, mood disorders, heart disease, mental illness, microbial infections, HIV, eye diseases, and combinations thereof.

6. (canceled)

7. The method of claim 1, wherein the cell membrane protein is selected from the group consisting of cell membrane receptors, G-protein coupled receptors (GPCRs), plasma membrane proteins, human beta 2 adrenergic receptor, viral receptors, Angiotensin Converting Enzyme 2, HER 2 receptors, or combinations thereof.

8. (canceled)

9. The method of claim 1,
wherein the first peptide comprises Small fragment of Nano Luciferase (SmBiT),
wherein the second peptide comprises Large fragment of Nano Luciferase (LgBit),
wherein SmBiT comprises SEQ ID NO:1 or a sequence that shares at least 65% sequence identity to SEQ ID NO:1, and
wherein LgBiT comprises SEQ ID NO:2 or a sequence that shares at least 65% sequence identity to SEQ ID NO:2.

10-11. (canceled).

12. The method of claim 1, wherein at least 90% of the flexible amino acid linker residues comprise glycine residues, serine residues, or combinations thereof.

13. The method of claim 1, wherein the second peptide comprises a flexible amino acid linker.

14. The method of claim 13, wherein the second peptide further comprises a cell membrane insertion domain, wherein the cell membrane insertion domain inserts onto an internalized cell membrane in a pH dependent manner, and wherein the cell membrane insertion domain is separated from the second peptide by the flexible amino acid linker.

15. The method of claims 1, wherein the flexible amino acid linker comprises a sequence selected from the group consisting of GSSGGGGSGGGGSSGGAQGNS (SEQ ID NO:3), GNSGSSGGGGSGGGGSSG (SEQ ID NO:4), GSSGGGGSGGGGSSG (SEQ ID NO:5), GSSGGGGSGGGGSSGGAQGNS (SEQ ID NO:6), a sequence that shares at least 65% sequence identity to any one of SEQ ID NOS: 3-6, or combinations thereof.

16. The method of claim 14, wherein the cell membrane insertion domain comprises a sequence of MQKQPTWVPDSEAPNCMNCQVKFTFTKRRHHCRACGKVFCGVCCNRKCKLQYLEKE ARVCVVCYETISK (SEQ ID NO: 7), or a sequence that shares at least 65% sequence identity to SEQ ID NO: 7.

17. The method of claim 1, wherein the flexible amino acid linker comprises at least 18 residues.

18. The method of claim 1, wherein the second peptide is expressed in the cell.

19. The method of claim 1, wherein the second peptide is added to the cell.

20. The method of claim 1, wherein the cells comprise live cells.

21. The method of claim 1, wherein the associating occurs by adding the binding agent to the cells.

22. The method of claim 1, wherein the detecting comprises detecting endocytosis of the cell membrane protein, detecting receptor-mediated endocytosis of the cell membrane protein, or combinations thereof.

23. (canceled)

24. The method of claim 1, wherein the detecting occurs in real-time.

25. The method of claim 1, wherein the detecting occurs by monitoring the internalization of the membrane protein through an increase in luminescence emitted by the cell membrane protein after internalization, wherein the increase in luminescence occurs when a first peptide fused to the cell membrane protein interacts with a second peptide associated with the internalized cell membrane or an early endosome marker.

26. The method of claim 25, wherein the luminescence comprises bioluminescence.

27. (canceled)

28. The method of claim 1, wherein a single binding agent is screened against a single type of cell membrane protein associated with the cell.

29. The method of claim 1, wherein a single binding agent is screened against a plurality of different types of cell membrane proteins associated with the cell.

30. The method of claim 1, wherein a plurality of different binding agents is screened against a single type of cell membrane protein associated with the cell.

31. The method of claim 1, wherein a plurality of different binding agents is screened against a plurality of different types of cell membrane proteins associated with the cell.

32. The method of claim 1, wherein the method is utilized to characterize the pharmacological properties of the binding agent.

33-48. (canceled).


SEQUENCE LISTING (7 sequences)

SEQ ID NO: 1 - protein, 11 aa, Artificial Sequence
  Small fragment of Nano Luciferase (SmBiT)
VTGYRLFEEIL

SEQ ID NO: 2 - protein, 158 aa, Artificial Sequence
  Large fragment of Nano Luciferase (LgBit)
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 3 - protein, 21 aa, Artificial Sequence
  Flexible Amino Acid Linker
GSSGGGGSGGGGSSGGAQGNS

SEQ ID NO: 4 - protein, 18 aa, Artificial Sequence
  Flexible Amino Acid Linker
GNSGSSGGGGSGGGGSSG

SEQ ID NO: 5 - protein, 15 aa, Artificial Sequence
  Flexible Amino Acid Linker
GSSGGGGSGGGGSSG

SEQ ID NO: 6 - protein, 21 aa, Artificial Sequence
  Flexible Amino Acid Linker
GSSGGGGSGGGGSSGGAQGNS

SEQ ID NO: 7 - protein, 69 aa, Artificial Sequence
  Cell Membrane Insertion Domain
MQKQPTWVPDSEAPNCMNCQVKFTFTKRRHHCRACGKVFCGVCCNRKCKLQYLEKEARVCVVCYETISK